(19) United States
(12) Patent Application Publication
(10) Pub. No.: US 2026/0273275 A1
(43) Pub. Date: Sep. 17, 2026

(54) System and Method for Treating a Patient by Applying an Electric Field

(71) Applicants: Scott Sheftel, Tucson, AZ (US); Jeffry Skiba, Chandler, AZ (US)

(72) Inventors: Scott Sheftel, Tucson, AZ (US); Jeffry Skiba, Chandler, AZ (US)

(21) Appl. No.: 19/672,444

(22) Filed: May 8, 2026

Related U.S. Application Data
(63) Continuation-in-part of application No. 18/131,777, filed on Apr. 6, 2023.
Application No. 18/131,777 is a continuation-in-part of application No. 17/920,177, filed on Oct. 20, 2022, now abandoned.
Application No. 17/920,177 is a continuation-in-part of application No. 15/823,076, filed on Nov. 27, 2017, now Pat. No. 11,124,901.
(60) Provisional application No. 63/802,089, filed on May 8, 2025.

(51) Int. Cl. A61N 1/20; A61N 1/04

(52) U.S. Cl. CPC A61N 1/20; A61N 1/0408

(57) ABSTRACT

A bioelectric device for treating a patient comprises a flexible substrate with strategically placed zinc reservoirs functioning as half-cells of a battery when contacting skin. The device operates through zinc's negative potential driving oxidation when exposed to perspiration and oxygen, generating therapeutic microcurrents without external power. Zinc particles are embedded rather than encapsulated, maintaining exposure while ensuring structural integrity. Clinical applications include moisture control, muscle fatigue relief, accelerated purpura resolution, skin health improvement, anti-aging benefits, enhanced iontophoresis, wound healing, and muscle stimulation. The device, implementable as wearable garments, maintains effectiveness through 40+ washing cycles, providing a self-sustaining approach to pain management, tissue regeneration, and dermatological treatment without requiring batteries, electronics, or professional supervision.

### BACKGROUND OF THE INVENTION

### Field of the Invention

The field of technology addressed by the disclosed subject matter pertains to bioelectric treatment devices and methods. More specifically, it involves systems and methods for applying controlled electric fields to a patient's skin using zinc-based electrochemical configurations. More specifically, it involves a system for producing microcurrents through a unique half-cell battery configuration where zinc reservoirs function as one half-cell (anode) while the patient's skin serves as the complementary half-cell (cathode), creating a complete galvanic circuit without external power sources. This technology integrates principles from electrochemistry, materials science, and biomedical engineering to create self-powered therapeutic devices that generate physiologically relevant electrical fields for various medical and cosmetic applications, including pain management, tissue regeneration, and treatment of dermatological conditions.

### Description of Related Art

Existing technologies in the sphere of skin treatments often rely on topical applications or invasive procedures, which may present challenges such as limited penetration of active ingredients or the need for complex equipment. In particular, traditional methods, including creams and ointments, struggle to achieve deep penetration through the epidermis and may require repeated applications to maintain effectiveness. Existing technologies for applying electric fields to biological tissues typically rely on externally powered devices that require batteries, complex circuitry, or connection to electrical outlets. These conventional electrotherapy systems often involve cumbersome equipment and electrode configurations that limit their practical use in everyday settings. Most commercial devices employ large surface electrodes that create non-uniform electrical fields which poorly mimic the body's natural bioelectric environment.

Traditional methods for electrical stimulation frequently demand professional supervision and cannot be easily integrated into patients' daily routines. While some portable devices exist, they generally require replacement batteries and complex user interfaces that create barriers to consistent use. Furthermore, current technologies typically cannot provide sustained electrical stimulation over extended periods without frequent intervention or maintenance.

In the realm of pain management and tissue regeneration, pharmaceuticals remain the predominant approach despite their potential side effects and limitations. Non-drug alternatives often fail to adequately address conditions like neuropathic pain, inflammatory disorders, or cosmetic concerns such as purpura. Additionally, existing electrical stimulation approaches struggle to penetrate the intact epithelial barrier to affect underlying tissues without invasive procedures.

Certain prior art devices have employed bimetallic galvanic configurations, wherein two dissimilar metals such as silver and zinc are deposited in alternating patterns on a substrate to generate localized electric fields upon contact with moisture. Such bimetallic systems are limited in several respects. Each metal pair operates as an isolated galvanic couple, restricting the spatial uniformity and total electrochemical capacity of the resulting field. The inclusion of a second metal such as silver introduces material cost, regulatory complexity, and potential for adverse biological effects including systemic metal accumulation, particularly in patients with compromised skin barriers. The surface-deposited nature of such printed bimetallic configurations further limits durability under repeated mechanical stress or laundering.

Additionally, because discrete electrode pairs in such systems are not electrically interconnected, each couple functions independently and the device cannot draw on the electrochemical capacity of the system as a whole. What is needed is a bioelectric device employing a single active metal in a galvanic configuration that uses tissue-derived moisture and oxygen as the complementary half-cell, wherein discrete metal reservoirs are electrically interconnected into a unified parallel architecture to provide spatially uniform field generation across the full treatment area and extended total electrochemical capacity, and wherein the active metal is integrated into the substrate in a manner that provides durability through extended wear and laundering.

### BRIEF SUMMARY OF THE INVENTION

The present invention provides among other things of an applicator that creates an electric field using a unique half-cell battery configuration. These devices feature zinc reservoirs applied to substrates such as collagen or textiles, with the zinc particles functioning as both electrodes and material reservoirs. Reservoirs of zinc, magnesium, or other suitable half-cell metal may be electrically interconnected through a conductive network comprising, for example, carbon nanotubes, graphene, conductive polymers, or other conductive elements that interconnect the reservoirs without themselves functioning as half-cells, creating a unified system where capacity increases while voltage remains constant, ensuring consistent therapeutic effects across the treatment area.

The invention has diverse applications including treating conditions like acute and chronic wounds, purpura, diabetic neuropathy, and joint pain through specially designed wearable textiles such as arm sleeves, socks, and compression garments. When applied to the skin, the zinc interacts with and is oxidized by moisture and oxygen from the body to generate an electrical field that can penetrate the intact epithelial barrier, disrupt pain signals by “jamming” afferent nerve transmissions, and provide therapeutic benefits to underlying tissues without pharmaceutical intervention.

The invention also provides an applicator comprising a substrate on which discrete reservoirs of a half-cell metal (such as zinc or magnesium) are arranged in isolated concentrations separated from neighboring reservoirs by an insulating region, gap, or other non-conductive separation. In particular embodiments, the substrate defines a topography of ridges and troughs and the reservoirs are placed on the ridges, with the troughs providing the insulating separation; in other embodiments the separation is provided by a dielectric material, a gap in the carrier, or other non-conductive region between reservoirs. When applied to the patient's skin, the reservoirs function as half-cells of a battery and, by virtue of their spaced and isolated arrangement, generate a designed matrix of electric fields across the treatment area. The strength of each field within the matrix may be substantially uniform across the array or may vary based on the spacing and geometry between neighboring reservoirs, allowing the matrix to be tuned for particular therapeutic applications.

The substrate may be formed from various materials including collagen, textiles, hydrogels, hydrocolloids, or starches. In some embodiments, the substrate may be derived from living organisms. The zinc reservoirs comprise zinc particles that are deposited onto the ridges of the substrate with desired spacing. These zinc reservoirs can be applied through solvent casting, wherein zinc powder is suspended in a cellulose solution to create a slurry that is then dropped or sprayed onto the substrate. In certain embodiments, the substrate may be engineered to release oxygen over time.

It is an object of the invention to provide electrical stimulation to a patient's skin without requiring external power sources or complex electrode configurations.

It is an object of the invention to provide a device for generating physiologically relevant electrical fields over living tissue through the use of distributed zinc-based half-cells and tissue moisture and oxygen as a complementary half-cell.

It is another object of the invention to provide a flexible substrate with printed patterns of zinc-containing compositions that can conform to tissue surfaces while maintaining consistent electrical field generation.

It is another object of the invention to provide a method of manufacturing wearable garments incorporating filaments containing a half-cell metal (such as zinc or magnesium) and a conductive network (such as carbon nanotubes, graphene, or conductive polymers) to create unified half-cell sources capable of generating therapeutic electrical fields.

It is another object of the invention to provide a system for controlled degradation of zinc-containing carriers to maintain consistent electrical field generation over extended periods of treatment.

It is another object of the invention to provide a method of downregulating matrix metalloprotease enzymes through the incorporation of therapeutic carriers such as polycellulose while simultaneously generating beneficial electrical fields.

It is another object of the invention to provide a device that creates electrical fields more closely matching natural transepithelial potentials than conventional large-electrode systems.

It is another object of the invention to provide a system combining zinc-based electrical field generation with supplemental oxygen delivery through the integration of oxygen-generating polymers.

It is another object of the invention to provide a method of electrically connecting discrete reservoirs of a half-cell metal (such as zinc or magnesium) using a conductive network of interconnecting elements (such as carbon nanotubes, graphene, or conductive polymers) to extend device electrical capacity and maintain consistent field generation.

It is another object of the invention to provide a device capable of modifying local cellular environments through controlled galvanotaxic effects and regulation of pH and reactive oxygen species levels.

It is another object of the invention to deliver collagen supplementation to targeted areas of a patient's skin while simultaneously providing electrical stimulation, wherein the electrical stimulation supports trophic factor release and endogenous collagen production.

It is a further object of the invention to provide a conformable electrical stimulation device that can adapt to various contours of a patient's body while allowing appropriate gas exchange.

It is another object of the invention to provide a system for enhancing cellular communication and tissue repair processes through the generation of controlled electrical fields at physiologically relevant levels.

It is another object of the invention to create a self-powered therapeutic device using biocompatible materials.

It is a further object of the invention to provide a device that can promote wound healing through both electrical stimulation, oxygen release and delivery of oxygen and nutrients to healing skin.

It is another object of the invention to provide a method for manufacturing an electrical stimulation device using simple solvent casting techniques.

It is a further object of the invention to provide localized electrical fields that can promote cellular migration and tissue regeneration.

It is another object of the invention to create a device that can aid in collagen stimulation and aid in scar reduction and management.

It is a further object of the invention to provide a device that can enhance transdermal drug delivery through iontophoresis.

It is another object of the invention to create a device that can promote hair growth through electrical stimulation to hair follicles and surrounding collagen.

It is a further object of the invention to provide a device that can aid in the treatment of muscle aches and fatigue through electrical stimulation.

It is another object of the invention to create a device that can enhance skin rejuvenation and anti-aging treatments via collagen stimulation.

It is a further object of the invention to provide a device that can stimulate circulation and aid in the treatment of inflammatory skin conditions.

It is another object of the invention to create a device that can promote bone healing in cases of fractures or osteoporosis.

It is a further object of the invention to provide a device that can enhance athletic recovery through localized electrical stimulation by stimulating circulation to enhance delivery of oxygen and nutrients.

It is another object of the invention to create a device that can aid in the treatment of chronic pain conditions by stimulating underlying nerves in the skin.

It is a further object of the invention to provide a device that can promote healing of diabetic ulcers through multiple therapeutic mechanisms via electrical stimulation of nerves.

It is another object of the invention to create a device that can enhance the effectiveness of physical therapy treatments.

It is a further object of the invention to provide a device that can aid in the treatment of peripheral nerve injuries.

It is another object of the invention to restore or improve peripheral nerve sensation in patients with diabetic peripheral neuropathy through bioelectric stimulation.

The present invention relates to bioelectric devices and methods for creating therapeutic electric fields on a patient's skin. In one aspect, the invention provides a bioelectric device comprising a flexible substrate, a plurality of half-cell metal reservoirs (for example, zinc or magnesium reservoirs) disposed on the substrate, and a conductive network electrically connecting these reservoirs. This conductive network may comprise carbon nanotubes, graphene, conductive polymers, or other conductive elements that interconnect the reservoirs without themselves functioning as half-cells, and is configured to provide electron mobility between reservoirs during galvanic reactions. The reservoirs may comprise particles of the half-cell metal dispersed within a degradable carrier material, with the conductive elements dispersed throughout this carrier material. The carbon nanotubes typically comprise between 0.1% and 10% by weight of the carrier material and have diameters between 10 and 50 nanometers and lengths between 1 and 10 micrometers. The degradable carrier material may comprise polycellulose.

The invention also provides a method of manufacturing such a bioelectric device by providing a flexible substrate, forming a composition comprising zinc particles, a conductive network, and a carrier material, and disposing this composition on the substrate to form electrically connected zinc reservoirs. The manufacturing process may involve dispersing carbon nanotubes in the carrier material at a concentration between 0.1% and 10% by weight.

A further aspect of the invention provides a bioelectric dressing comprising a flexible substrate configured to conform to a tissue surface, discrete repositories of a half-cell metal (such as zinc or magnesium) disposed in a predetermined pattern, a carrier material containing these repositories, and a conductive network dispersed through the carrier material, the conductive network comprising carbon nanotubes, graphene, conductive polymers, or other conductive elements. This conductive network maintains electrical connectivity between the repositories as the carrier material degrades. The carrier material may comprise a therapeutic agent, specifically polycellulose configured to downregulate matrix metalloprotease enzymes. The dressing may further incorporate an oxygen-generating polymer integrated with the carrier material.

The invention additionally provides a wearable bioelectric device comprising a fabric substrate with filaments containing a half-cell metal (such as zinc or magnesium) integrated into it and conductive elements (such as carbon nanotubes, graphene, or conductive polymers) incorporated into these filaments to create a conductive network throughout the fabric. The zinc-containing filaments may comprise zinc particles dispersed within a polymeric carrier and may be woven into the fabric substrate.

A method of providing bioelectric treatment is also disclosed, comprising applying to a tissue surface a flexible device with reservoirs of a half-cell metal (such as zinc or magnesium) electrically connected by a conductive network (such as carbon nanotubes, graphene, or conductive polymers), generating electrical fields through galvanic coupling between the reservoirs and moisture and oxygen from the tissue surface, and maintaining these fields through electron mobility provided by the conductive network as the carrier material degrades. The electrical fields typically have a potential of approximately 0.17 to 1 volt. The method may further include supplying supplemental oxygen through an oxygen-generating polymer integrated into the device.

Another embodiment of the invention provides an applicator for creating a sustained electric field, comprising a textile composed of woven fibers with embedded zinc reservoirs and a linking component that electrically connects these reservoirs to provide electron mobility during galvanic reactions.

The invention further includes a method of treating conditions such as purpura, tennis (or golfer's) elbow, convalescence aches, diabetic neuropathy, and arthritic conditions. This method involves forming fibers with embedded zinc reservoirs, forming a textile from these fibers, and applying the textile to the patient's skin near the condition to initiate a galvanic reaction that creates a therapeutic electric field. The textile may take the form of a sleeve, arm or leg band, sock, compression shirt, compression shorts, or compression pants.

Another aspect of the invention includes an applicator comprising a substrate on which discrete reservoirs of a half-cell metal (such as zinc or magnesium) are arranged in isolated concentrations such that neighboring reservoirs are electrically isolated and spaced from one another by an insulating region, gap, or other non-conductive separation. In a particular embodiment the substrate defines a plurality of ridges and troughs and the reservoirs are deposited on the ridges with the troughs providing the insulating separation. When applied to a patient's skin, the reservoirs form half-cells of a battery that creates a beneficial electric field, and the spaced isolated arrangement produces a designed matrix of electric fields across the treatment area, the strengths of which may be uniform or may vary based on inter-reservoir spacing. The substrate may be grown from a living organism, specifically collagen, or may alternatively be a textile. The reservoirs may consist of particles of the half-cell metal deposited onto the substrate.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112(f). Moreover, even if the provisions of 35 U.S.C. § 112(f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts a perspective view of an applicator according to one or more embodiments of the invention.

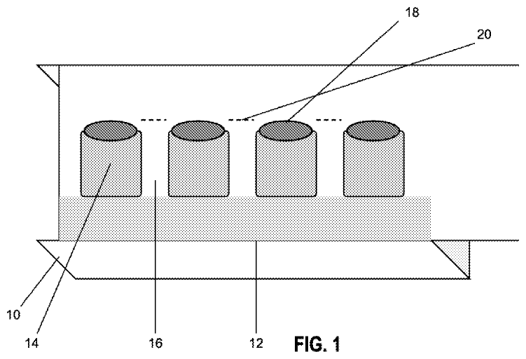

FIG. 2 depicts a cross-sectional view of the applicator of FIG. 1 applied to a patient's skin according to one or more embodiments of the invention.

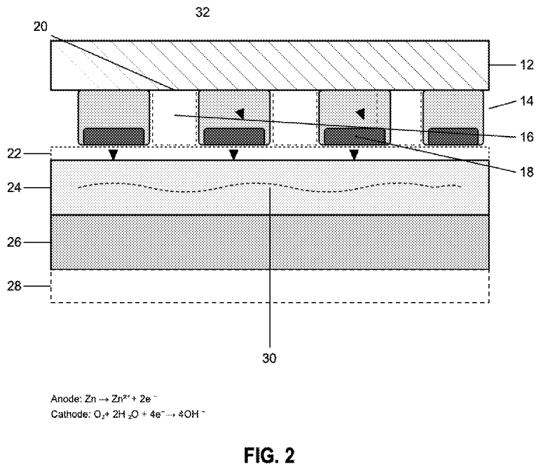

FIG. 3 depicts diagrammatic views of a textile embodiment according to one or more embodiments of the invention.

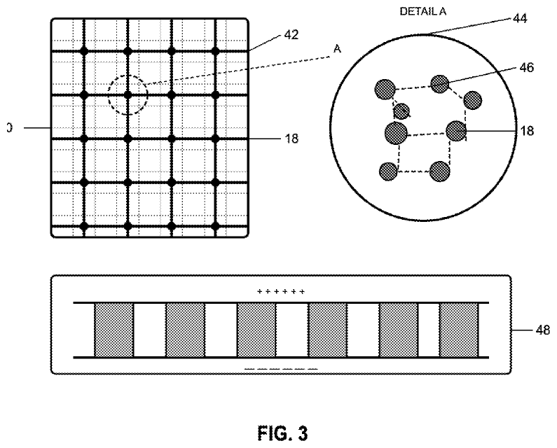

FIG. 4 depicts a flow diagram of a method of manufacturing and applying a bioelectric applicator according to one or more embodiments of the invention.

FIG. 5 depicts a section view of a collagen substrate according to one or more embodiments of the invention.

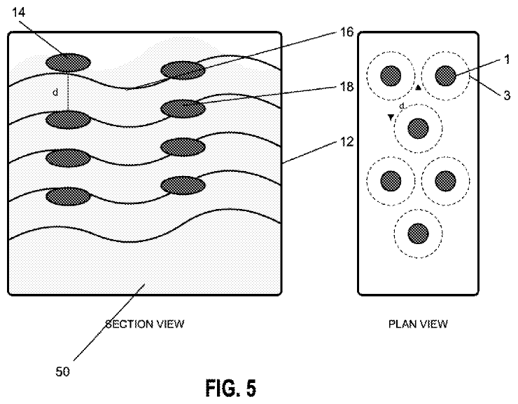

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

The invention relates to devices and methods for providing exogenous electrical field matrices over living tissue surfaces through the use of electrically interconnected zinc reservoirs 18.

The present invention provides a system for creating therapeutic electrical fields that leverage fundamental biological principles wherein human cells demonstrate sensitivity to electrical fields. The shape, amplitude, duration, and frequency of these electrical fields can significantly impact cellular responses, influencing functions such as nerve signal transmission and various cellular activities regulated through voltage-dependent gates located throughout cell membranes.

The human body naturally creates electric fields at very low levels as part of normal metabolic processes. As cells metabolize ATP, converting it into carbon dioxide, concentration gradients develop across cell membranes, contributing to the generation of these natural electric fields. The invention described herein provides methods of creating exogenous electric fields that supplement these natural processes, with the capacity to cover affected areas in a controlled manner through the strategic placement of zinc-containing reservoirs and their electrical interconnection, in effect generating and mimicking the electric field of the body.

The invention leverages fundamental biological principles wherein human cells demonstrate sensitivity to electrical fields, with many physiological activities being field-dependent. These include muscle contraction, nerve signal transmission, and various cellular functions regulated through voltage-dependent gates located throughout cell membranes. The shape, amplitude, duration and frequency of electrical fields can significantly impact cellular response.

The electrical fields generated by the present invention produce several subclinical therapeutic effects that contribute to its overall efficacy. At the cellular and tissue level, the zinc-oxygen battery configuration creates microcurrents that enhance local blood flow to affected areas, facilitating improved oxygenation and nutrient delivery while promoting waste product removal. Additionally, these microcurrents create an environment that inhibits the proliferation of pathogenic organisms including bacteria, fungi, and viruses, providing a protective effect without pharmaceutical antimicrobials. Of particular significance is the stimulation of collagen synthesis within the dermal layer, which occurs even when the device is applied to intact skin. This enhanced collagen production contributes to improved skin integrity, accelerated healing processes, and cosmetic benefits. The electrical field further promotes the expression and release of various trophic factors-specialized signaling molecules that regulate cellular growth, differentiation, and tissue maintenance. These trophic factors play crucial roles in tissue regeneration pathways and contribute to the sustained therapeutic benefits observed following treatment with the device. The combined action of these subclinical effects creates a comprehensive therapeutic response that addresses multiple aspects of tissue health and function.

The invention utilizes zinc as an active electrode material in conjunction with moisture and oxygen derived from a patient's body to form an electrochemical cell. When placed on the skin, the zinc in the reservoirs interacts with moisture and oxygen naturally present in the patient's skin to create a zinc-oxygen battery effect. This configuration produces electrical fields of approximately 0.17 to 1 volt, matching physiologically relevant levels. The moisture layer 22 and oxygen (cathode 24) from the body serves as one half-cell of the battery, while the zinc reservoirs serve as the other half-cell (anode), creating a complete electrochemical system without requiring external power sources.

The tissue-device interface is engineered to facilitate ion exchange between the device and tissue, maintain appropriate moisture levels, ensure consistent electrical contact, and allow for gas exchange while maintaining electrical properties. The generated electrical fields influence multiple cellular mechanisms including regulation of voltage-dependent gates in cell membranes, modification of cellular migration patterns, influence on cellular communication, modulation of transepithelial potential, and enhancement of trophic factor expression.

A significant aspect of the present invention involves its therapeutic applications for various conditions. The electrical field generated by the zinc-based material is capable of penetrating through the epithelial layer of the skin to reach underlying tissues. The epidermis 24 typically comprises multiple layers of cells, including non-living cells on the surface, forming a barrier that conventional treatments struggle to penetrate. The electrical field generated by the present invention can penetrate this barrier to deliver therapeutic effects to the dermis 26 and deeper tissues.

The present invention demonstrates a diverse range of clinical effects that provide therapeutic benefits across multiple applications. When applied to the skin, the electrical field generated by the zinc-oxygen battery system actively contributes to moisture and odor control, creating a healthier microenvironment in areas prone to excessive perspiration. Patients experiencing muscle fatigue, joint stiffness, or discomfort report significant relief following application, likely due to the electrical field's interference with pain signal transmission and enhanced cellular recovery mechanisms. The device shows efficacy in managing sweat-related rashes and general skin health concerns by modulating the local environment and supporting natural barrier functions. Anti-aging benefits have been observed through improved collagen synthesis and cellular metabolism. The electrical field facilitates iontophoresis, enhancing transdermal delivery of beneficial compounds when used in conjunction with topical applications. In wound care applications, the device promotes accelerated healing through multiple pathways including enhanced cellular migration and antimicrobial effects. Additionally, the specific electrical parameters generated by the invention provide gentle stimulation to fast-twitch muscle fibers, potentially contributing to improved muscle tone and function when applied over extended periods.

One unexpected application of the invention is in the treatment of purpura, a condition characterized by discoloration under the skin due to bleeding from small blood vessels. Unlike wound healing applications where the skin barrier is compromised, purpura represents an intact skin barrier where the therapeutic target lies beneath the epidermis. This application falls within the cosmeceutical or cosmetic category rather than wound healing, as it addresses discoloration without any disruption of the epithelium. The invention's effectiveness for this application was discovered when the material was applied over patients experiencing purpura, and unexpectedly resulted in improvement of the condition.

Another significant application is in pain management for various conditions including diabetic neuropathy, tennis elbow and other tendinopathies, arthritic conditions, generalized aches and pains, and post-exercise recovery. The mechanism for pain relief involves the disruption of pain signals transmitted by afferent nerve fibers in the skin. The electrical field generated by the invention interferes with these signals, effectively “jamming” the pain transmission to the brain's pain centers. Afferent nerve fibers carry signals to the central nervous system, and when an electrical field is applied over these electrical conductors, it can disrupt the signal, similar to jamming radio signals. This disruption results in reduced pain perception without the need for pharmaceutical interventions. This application is particularly notable for diabetic patients, who often suffer from persistent extremity pain that is difficult to treat with conventional methods. In addition to reducing neuropathic pain, the electrical field generated by the invention has been observed to enhance peripheral nerve sensitivity and tactile sensation in diabetic patients, where chronic hyperglycemia has impaired sensory nerve function.

Beyond superficial applications, the invention provides therapeutic benefits to deeper tissues including joints affected by inflammatory conditions, muscles experiencing fatigue or strain, and connective tissues subject to injury or deterioration. The electrical field generated by the zinc-based material penetrates beyond the skin surface to affect underlying tissues, promoting improved circulation, reduced inflammation, and enhanced tissue recovery. These effects occur without needing to breach the skin barrier, providing a non-invasive treatment option for deep tissue conditions.

In accordance with one aspect of the present invention, the device comprises a flexible substrate 12 with zinc reservoirs. Zinc reservoirs may be electrically interconnected and distributed across a substrate 12. The zinc reservoirs are dispersed on the substrate 12 in a pattern of discrete isolated locations. These reservoirs may be electrically interconnected to create a matrix of electric fields 30 to function as a unified system.

When zinc particles at the tissue interface oxidize during the galvanic reaction, the resulting charge imbalance is rapidly equilibrated through electron flow 32 along the conductive network 46 (which may comprise, for example, carbon nanotubes, graphene, or conductive polymers). This equilibration draws electrons from particles of the half-cell metal throughout the device, effectively making all zinc reservoirs available for the galvanic reaction regardless of their proximity to the tissue interface. The high conductivity of the carbon nanotube network ensures that this charge redistribution occurs with minimal resistive losses, maintaining the desired electrical potential at the tissue interface. When the zinc reservoirs are electrically interconnected, they operate in parallel, creating a system where the electrical capacity of the overall electrochemical system increases while the voltage remains constant. This parallel configuration ensures that regardless of which portion of the material contacts the skin, the user receives the benefit of the entire system's capacity.

The zinc reservoirs serve dual purposes: as electrodes that conduct the electrical field and as material reservoirs that hold the capacity of the battery-like system. The interconnected configuration extends treatment duration beyond what would be possible with isolated zinc deposits, as the entire mass of zinc contained within the device becomes available for the galvanic reaction, rather than being limited to only the zinc in direct contact with the tissue interface.

The zinc compositions include zinc powder combined with a therapeutic carrier 44 such as polycellulose, with controlled zinc particle sizes and distributions. The carriers are designed for controlled degradation to gradually expose the half-cell metal and may integrate conductive elements such as carbon nanotubes, graphene, or conductive polymers for enhanced electrical connectivity between the metal particles. The polycellulose carrier provides dual functionality by both binding the zinc particles and downregulating matrix metalloprotease (MMP) enzymes that can impair healing in chronic wounds.

More generally, the carrier material may comprise one or more materials selected from the group consisting of polycellulose, carboxymethylcellulose, hydroxypropyl cellulose, hydroxyethyl cellulose, alginate, chitosan, collagen, gelatin, hyaluronic acid, starch, polyvinyl alcohol, polylactic acid, poly(lactic-co-glycolic acid), and combinations thereof, each selected for its ability to bind zinc particles, control degradation rate, and provide therapeutic modulation of the wound microenvironment including downregulation of matrix metalloprotease enzymes.

In another aspect of the present invention, the device comprises specialized fabric incorporating zinc-containing filaments 42 woven into flexible garments. These may include round or flat wires of a half-cell metal (such as zinc or magnesium) with optimized diameters and base filaments enhanced with conductive elements (for example, carbon nanotubes, graphene, or conductive polymers) for improved conductivity. The zinc elements are interconnected to function as a unified half-cell source, providing enhanced capacity and consistent field generation.

The invention may be implemented in various forms, including arm or knee sleeves, socks, compression garments, therapeutic wraps, or other textile products. The specific embodiments described below illustrate the invention but do not limit its scope.

The pattern geometry and spacing of the zinc reservoirs can be precisely adjusted to optimize the electrical field distribution for specific therapeutic applications. Different patterns may be employed for different treatment areas or conditions to maximize therapeutic efficacy. The patterned application of zinc allows for controlled electrical field characteristics that cannot be achieved with random or uniform zinc distribution as would occur with zinc ointments or similar preparations. A random zinc distribution will not generate an electric field.

The substrate 12 may be implemented using various materials, including but not limited to textiles 40, fabrics, collagen, hydrogels, hydrocolloids, starches, or combinations thereof. The substrate 12 materials may comprise various polymers, including recycled polymers, that provide structural support while allowing proper electrochemical interactions. The substrate 12 may be flexible to conform to body contours or rigid for specific applications. In some embodiments, the substrate 12 may be derived from living organisms. When applied to textiles or fabrics with natural surface variations, the zinc material may follow the topography of the substrate 12, creating a naturally ridged pattern that follows the underlying structure. This ridged configuration may provide enhanced contact with the skin surface and improved electrical conductivity.

In a particular embodiment, zinc particles are embedded in the substrate 12 rather than encapsulated, ensuring that a portion of each zinc particle remains exposed to the electrochemical environment while being securely anchored to the carrier material. This configuration allows for optimal oxidation of the zinc while maintaining the structural integrity of the device. When incorporated into textiles, the zinc-embedded materials maintain therapeutic effectiveness through more than 40 standard washing cycles, consistently producing beneficial microcurrents throughout this extended lifespan. This exceptional durability enables long-term use in everyday garments and medical textiles, significantly enhancing the cost-effectiveness and practical utility of the technology for ongoing therapeutic applications. The persistence of the electrochemical activity after repeated washing cycles demonstrates the robust integration of the zinc reservoirs within the substrate 12 matrix and the stability of the conductive network that electrically connects these reservoirs.

A further embodiment of the invention combines zinc-based electrical field generation with supplemental oxygen delivery through integration of oxygen-generating polymers. The oxygen release is controlled to enhance zinc-oxygen battery effects, with optimized spatial arrangement of oxygen-generating and zinc-containing components. Both oxygen and zinc sources feature tuned degradation profiles to maintain consistent field generation throughout the therapeutic period. The inherent standard potential of the zinc determines the galvanic potential delivered.

The invention generates electrical fields through galvanic coupling between zinc half-cells, and moisture and oxygen from tissue respiration acting as the second half-cell. Zinc possesses a standard reduction potential of −0.76V versus the standard hydrogen electrode, which is significantly negative compared to many other metals. This negative potential creates a strong driving force for oxidation when zinc is placed in an environment containing oxygen and electrolytes, such as those found in human perspiration. This creates ideal conditions for the zinc oxidation reaction: Zn→Zn²⁺+2e⁻. The liberation of these electrons creates the electrical current that flows through the tissue. Simultaneously, oxygen reduction occurs at the skin interface: O₂+2H₂O+4e⁻→4OH⁻, completing the electrochemical circuit.

This redox couple generates approximately 0.17 to 1 volt of potential difference, which matches the physiologically relevant range for bioelectric effects without causing discomfort or tissue damage. The rate of zinc oxidation is naturally regulated by the availability of oxygen and moisture at the skin interface. During periods of increased perspiration, such as during physical activity, the heat, increased moisture and electrolyte content accelerates the electrochemical reaction, providing enhanced electrical output precisely when increased therapeutic effect may be beneficial. This self-regulating aspect represents a significant advantage over conventional electrical stimulation devices that deliver fixed outputs regardless of physiological conditions.

The gradual oxidation of zinc particles over time ensures a controlled release of electrical energy (creating a corresponding electric field) and zinc ions, both of which contribute to the therapeutic effects. The zinc ions themselves may provide additional benefits beyond the electrical effects, as zinc is known to have anti-inflammatory and wound healing properties. This antimicrobial activity of zinc ions at sub-lethal concentrations, known as the oligodynamic effect, operates through multiple mechanisms including disruption of bacterial cell membranes, inhibition of bacterial enzyme systems, and interference with biofilm formation, and is synergistic with the direct bioelectric antimicrobial effect of the generated electric field, providing a dual-mechanism antimicrobial action that is particularly advantageous in wound care applications where biofilm colonization is a primary barrier to healing. The sustained nature of this electrochemical reaction, powered by the negative standard potential of zinc, enables the device to maintain therapeutic effectiveness through extended wear periods and, remarkably, to retain functionality even after more than 40 standard washing cycles when incorporated into textile applications

The conductive network that electrically interconnects the zinc reservoirs may comprise one or more conductive elements including carbon nanotubes, graphene, reduced graphene oxide, carbon black, carbon fibers, silver nanowires, copper nanowires, conductive polymers such as poly(3,4-ethylenedioxythiophene) polystyrene sulfonate (PEDOT:PSS), polyaniline, or polypyrrole, MXenes, or combinations thereof. The conductive element is selected based on biocompatibility, compatibility with the manufacturing process, mechanical flexibility, and the desired electrical conductivity of the resulting device. Carbon nanotubes represent a preferred conductive element in certain embodiments by virtue of their high electrical conductivity, mechanical strength, and ability to form percolating networks at low concentrations within the carrier material

Carbon nanotubes are incorporated to electrically connect discrete zinc repositories, creating a unified half-cell source that extends the functional electrical capacity of the device while maintaining consistent field generation across the treatment area.

The conductive elements (which may comprise carbon nanotubes, graphene, conductive polymers, or combinations thereof) are incorporated into the carrier material in a concentration sufficient to create a percolating conductive network throughout the device structure. This network provides multiple parallel conductive pathways between zinc reservoirs. When zinc particles in direct contact with the tissue interface participate in the galvanic reaction, electrons are conducted through the carbon nanotube network to maintain electrical equilibrium across all zinc reservoirs in the device. This electron mobility enables zinc particles located distally from the tissue interface to contribute electrons to the galvanic reaction occurring at the interface.

In particular embodiments, the conductive elements comprise multi-walled carbon nanotubes dispersed throughout the carrier material at concentrations between 0.1% and 10% by weight; in other embodiments the conductive elements comprise graphene flakes, conductive polymers, or combinations of these materials at comparable loadings. The carbon nanotubes have typical diameters between 10 and 50 nanometers and lengths between 1 and 10 micrometers. These dimensions and concentrations are selected to ensure formation of a continuous conductive network while maintaining appropriate mechanical properties of the carrier material.

The incorporation of the conductive network further allows for the design of devices with varying densities and distributions of the half-cell metal reservoirs while maintaining consistent electrical performance. This flexibility in design enables optimization of reservoir placement for specific therapeutic applications without compromising the electrical functionality of the device. The carbon nanotube network ensures that all zinc reservoirs contribute to the galvanic reaction regardless of their spatial distribution within the device.

Parameters for manufacturing include zinc particle size distribution, carrier composition uniformity, electrical connectivity verification, and degradation rate validation. In fiber implementations, the zinc material may be incorporated into the fiber structure itself, either through extrusion processes where zinc particles are mixed with the fiber material prior to extrusion, or through post-extrusion coating processes. When zinc particles are incorporated within polyethylene or other thermoplastic fibers, the particles may represent between 3-70 volume percent of the fiber material. In a particular embodiment, the particles represents 3-5 wt % and are less than about 5 microns in size. These smart fibers can be woven, knitted, or otherwise formed into garments, compression wear, bandages, or other textile products that contact the skin.

The size of zinc particles used in the present invention plays a role in both manufacturing processes and device performance. Elemental zinc or zinc oxide particles smaller than 5 microns are specifically selected to prevent clumping and aggregation during the extrusion process when incorporating zinc into fibers. This small particle size ensures homogeneous distribution throughout the polymer matrix during fiber formation, preventing the formation of large zinc clusters that could disrupt the fiber structure or create weak points susceptible to breakage. When zinc particles exceed 5 microns, they tend to agglomerate during the high-pressure extrusion process, creating uneven distribution, potential clogging of extrusion nozzles, and inconsistent electrical properties in the final product. The sub-5-micron particle size also provides a good surface area for electrochemical reactions, enhancing the efficiency of the oxidation process when the fibers contact skin perspiration and oxygen. Additionally, these smaller particles integrate more seamlessly with the conductive network (whether comprising carbon nanotubes, graphene, conductive polymers, or other conductive elements), ensuring efficient electron mobility between the metal reservoirs throughout the fiber structure.

The concentration of zinc particles within the fiber matrix must be carefully controlled for desired electrical and mechanical properties. Experimental testing has determined that a zinc concentration of approximately 3-10% by weight provides the ideal balance between electrochemical performance and structural integrity. At this concentration range, the zinc particles are sufficiently abundant to generate therapeutic electrical fields while remaining properly dispersed throughout the polymer matrix. When zinc concentrations exceed 10% by weight, the resulting fibers become increasingly brittle and prone to fracture during manufacturing processes and normal wear. This brittleness occurs because higher zinc concentrations disrupt the continuity of the polymer chains, reducing their ability to flex and elongate under stress. Conversely, zinc concentrations below 3% by weight fail to produce sufficient electrical output for therapeutic efficacy. The 3-10% concentration range ensures that fibers maintain appropriate tensile strength, flexibility, and durability while providing consistent electrical performance throughout the intended product lifespan.

Zinc fibers incorporated in an electrically connected network may be determined through capacity testing. By calculating the molar equivalent of zinc present in visible reservoirs and comparing the theoretical duration of operation to the actual duration, interconnection can be determined. For example, if a single visible reservoir should theoretically provide electrical activity for two minutes based on its zinc content, but the system continues to function for four hours, this would indicate that the reservoirs are interconnected and sharing capacity. This testing method provides a practical means of identifying products that implement the electrically interconnected reservoir configuration.

The invention may be manufactured through various processes, including printing processes where zinc-containing materials are deposited in specific patterns, extrusion processes where zinc particles are incorporated into fibers, coating processes where zinc materials are applied to existing substrates 12, or electrochemical deposition methods. The zinc reservoirs may be formed through solvent casting, wherein zinc powder is suspended in a cellulose solution to create a slurry that is then dropped or sprayed onto the substrate 12. The slurry typically contains zinc particles that are about 2.5 to 8 microns in size, suspended at approximately 12% by weight zinc. In a particular embodiment, the zinc particles are under 5 microns in size. This slurry can be precisely applied to the substrate 12 using a measured applicator equipped with a small dispenser needle, allowing for controlled deposition of small amounts onto the surface.

In one application of the invention, provides an applicator for creating an electric field on the skin of a patient. In a particular embodiment, the applicator comprises a substrate 12 that defines a plurality of ridges 14 and troughs 16, with reservoirs of a half-cell metal (such as zinc or magnesium) strategically applied to the ridges 14 in a manner that ensures electrical isolation and spacing between neighboring reservoirs.

The substrate 12 may be formed from various biocompatible materials. In one embodiment, the substrate 12 is grown from a living organism, specifically collagen. Alternative embodiments utilize textile materials, chitosan, alginate, cellulose, silk fibroin, hydrogels, hydrocolloids, starches, or combinations as the substrate 12 material. The substrate 12 may be engineered to release oxygen over time, providing additional therapeutic benefits. For example, sodium percarbonate materials, calcium peroxide matrices, silicone-based oxygen carriers, glucose oxidase systems, oxygen-loaded microspheres, gas-entrapping hydrogels, oxygen-saturated lipid vesicles or porous materials with encapsulated oxygen-generating compounds may be used.

The zinc reservoirs consist of zinc particles that are deposited onto the ridges 14 of the substrate 12. In a preferred embodiment, the zinc particles are suspended in a polycellulose solution at approximately 12% by weight zinc to create a slurry. The particle size of the zinc may be about 2.5 to 4 microns, 3.5-5 microns, or 5-8 microns in size. This slurry can be precisely applied to the substrate 12 using measured applicator equipped with a small dispenser needle, allowing for controlled deposition of small amounts onto the surface.

Experimental testing has demonstrated the effectiveness of the applicator. When tested with water without added salt, the device generates approximately 0.17-0.6 volts. When applied to skin, the initial voltage measures approximately 0.17-0.6 volts, maintaining approximately 0.2-0.4 volts after two hours of application. Microscopic examination reveals that the polycellulose binder does not plate the entire surface of the substrate 12, but rather allows the zinc particles to adhere primarily to the fibers of the substrate 12.

The application process has been optimized for practical use. The zinc-containing slurry demonstrates fast absorption into the substrate 12 with moderate spreading characteristics, providing acceptable coverage of the target areas. When applying the slurry, small amounts are preferred as they maintain better position control compared to more generous applications which may exhibit additional spreading.

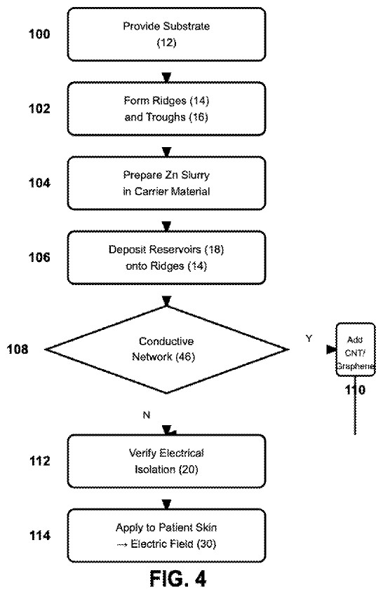

In accordance with another aspect of the invention, and with reference to FIG. 4, a method of providing a half-cell battery to a patient is described. The method begins by providing a substrate 12 (step 100) that is configured to support insulating separations 20 between the locations at which reservoirs 18 of a half-cell metal will reside. In one embodiment, a plurality of ridges 14 and troughs 16 are formed in the substrate 12 (step 102), with the troughs 16 providing the insulating separation 20 between adjacent ridges 14. The ridges 14 and troughs 16 may be formed by molding, embossing, weaving, knitting, three-dimensional printing, or any other technique suitable for the substrate material selected. The substrate 12 itself may be grown from a living organism, may comprise collagen, or may comprise a textile 40, hydrogel, hydrocolloid, starch, or combinations thereof. In embodiments where the substrate 12 is a hydrogel, hydrocolloid, or starch, the substrate 12 may be configured to release oxygen over time to enhance the galvanic reaction at the tissue interface.

A zinc slurry is then prepared by suspending zinc powder in a carrier material 44 (step 104) such as a polycellulose solution, carboxymethylcellulose, hydroxypropyl cellulose, or other cellulose-based or biocompatible liquid carrier. In a preferred embodiment, the zinc powder is suspended in a cellulose solution to facilitate uniform deposition and to provide controlled release characteristics. The reservoirs 18 are then deposited onto the substrate 12 in a spaced arrangement (step 106), preferably onto the ridges 14, such that neighboring reservoirs 18 are electrically isolated from one another by the insulating separation 20. Deposition may be performed by solvent casting, in which the slurry is dropped or sprayed onto the substrate 12 and the carrier liquid is allowed to evaporate, leaving the zinc particles adhered to the substrate 12 in discrete reservoirs 18. Other suitable deposition techniques include printing, dispensing through a metered applicator, dipping, and spraying.

At decision step 108, the method optionally includes incorporating a conductive network 46 between the reservoirs 18. Where electrical interconnection of reservoirs 18 is desired (the “Y” branch), conductive elements such as carbon nanotubes, graphene, or conductive polymers are added to the carrier material 44 or otherwise distributed across the substrate 12 (step 110), forming a conductive network 46 that links the reservoirs 18 while remaining distinct from the half-cell reservoirs themselves. Where interconnection is not desired (the “N” branch), this step is omitted and the reservoirs 18 operate as electrically independent half-cells. In either branch, the method then includes verifying the electrical isolation 20 between adjacent reservoirs 18 (step 112) to confirm that each reservoir 18, or each interconnected group of reservoirs 18, can function as an independent half-cell when in contact with the patient.

Finally, the substrate 12 with the deposited reservoirs 18 is applied to the skin of the patient (step 114) such that the reservoirs 18 form half-cells of a battery and the skin, together with the moisture layer 22 and oxygen at the skin surface, forms the opposing half-cell. The galvanic reaction between the reservoirs 18 and the skin generates a matrix of electric fields 30 across the treatment area, with each field having a strength determined by the standard potential of the half-cell metal and the spacing and geometry between neighboring reservoirs 18. In an embodiment directed to collagen supplementation, the substrate 12 is a collagen substrate and is applied to a location on the patient where collagen supplementation is desired, so that the electric field 30 promotes endogenous collagen production at that site while the substrate 12 itself serves as an exogenous collagen source.

In another aspect of the invention, a method of treating a condition selected from at least one of purpura, joint pain, tennis elbow, convalescence aches, diabetic neuropathy, and arthritic conditions is described. The method comprises forming a plurality of fibers having a plurality of a half-cell metal (such as zinc or magnesium) embedded therein, the embedded metal forming Zn filaments 42 or analogous filaments distributed along the length of each fiber. The fibers are then formed into a textile 40 by weaving, knitting, braiding, nonwoven processing, or other textile-forming techniques known in the art. The textile 40 is then applied to the skin of the patient in the vicinity of the condition, initiating a galvanic reaction between the embedded half-cell metal and the moisture and oxygen at the skin surface to create an electric field 30 at the treatment site.

The textile 40 may take the form of a sleeve, an armband, a leg band, a sock, a shirt, shorts, pants, or any other garment configured to be worn against the skin in the vicinity of the condition to be treated, and any such garment may be configured as either a compression garment or a non-compression garment depending on the therapeutic objective. In a further embodiment, the method includes forming a conductive network 46 within the fibers, for example by incorporating carbon nanotubes, graphene, or conductive polymers into the fiber matrix during fiber formation. Where carbon nanotubes are used, the carbon nanotubes preferably comprise between 0.1% and 10% by weight of the fibers, an amount sufficient to provide electrical interconnection between the embedded half-cell metal particles without compromising the mechanical or tactile properties of the resulting textile 40. The fibers themselves may comprise one or more materials selected from the group consisting of polycellulose, carboxymethylcellulose, hydroxypropyl cellulose, alginate, chitosan, collagen, gelatin, hyaluronic acid, polyvinyl alcohol, polylactic acid, and combinations thereof.

Where the substrate 12 defines a topography of ridges 14 and troughs 16, the troughs 16 create physical and electrical separation between neighboring reservoirs, providing one example of the insulating separation 20 that may be used to keep adjacent reservoirs electrically isolated; in other embodiments, the insulating separation may comprise a dielectric layer, a non-conductive polymer region, or a physical gap in the carrier material. In each case, the spaced and isolated arrangement of reservoirs produces a designed matrix of electric fields across the treatment area, the strength of each field within the matrix being substantially uniform or varying based on the spacing and geometry between neighboring reservoirs. The original zinc reservoirs on adjacent ridges 14, ensuring that each reservoir can function as an independent half-cell of the resulting battery when in contact with the patient's skin. This arrangement enables the creation of a distributed electric field across the treatment area.

In preferred embodiments, the collagen substrate 12 is maintained in the bottom part of a resealable package, allowing for repeated use while maintaining sterility and moisture content. Testing has confirmed that the package can be effectively resealed using a heat sealer between treatments.

The invention provides particular advantages in practical application. No conductive medium is required for skin application, though one may be needed for testing the printed substrate 12. The voltage generation is stable and consistent across treatment sites, and the physical format allows for precise placement on areas requiring treatment. The slurry may be mixed to maintain homogeneity, and precise dispensing techniques used for optimal application.

The invention may be deployed in a variety of embodiments. An arm sleeve for tennis elbow represents one embodiment of the invention. A sleeve according to one embodiment of the invention is shown in FIG. 7. The arm sleeve is fabricated from elastic fabric with zinc reservoirs printed in an evenly spaced pattern of discrete isolated locations on the inner surface. The reservoirs of half-cell metal (such as zinc or magnesium) may be electrically interconnected through a conductive network of carbon nanotubes, graphene, conductive polymers, or other conductive elements incorporated into the carrier material, although not all embodiments include such interconnection. When worn, the arm sleeve provides continuous electrical stimulation to the affected area, increasing blood flow, disrupting pain signals, and promoting tissue recovery.

Diabetic socks represent another embodiment. The socks are manufactured with fibers containing zinc particles that form interconnected reservoirs throughout the material. In some embodiments, the interconnection is achieved through a conductive network of carbon nanotubes, graphene, conductive polymers, or other conductive elements incorporated into the fiber matrix, although not all embodiments include such interconnection. The socks provide electrical stimulation to the feet and lower extremities of diabetic patients, reducing neuropathic pain and improving comfort during daily activities. A sock according to one embodiment of the invention is shown in FIG. 8.

A purpura treatment garment represents a third embodiment. This lightweight textile garment features precisely patterned zinc reservoirs designed to be worn over areas affected by purpura. The electrical field penetrates through the intact epidermis to reach the dermis where discoloration occurs, promoting more rapid resolution of the condition.

The present invention provides several advantages over existing technologies. The reservoir configuration, with or without electrical interconnection between reservoirs, extends treatment duration beyond what would be possible with isolated zinc deposits. The patterned application of zinc allows for controlled electrical field characteristics that cannot be achieved with random or uniform zinc distribution. The incorporation into everyday textiles and garments improves patient compliance compared to medical devices or bandages. The ability to penetrate the intact skin barrier allows for treatment of conditions that would not respond to topical applications. The use of the patient's own moisture and oxygen as a reactant simplifies the system and eliminates the need for complex battery structures. The parallel electrical configuration ensures consistent therapeutic effect regardless of which portion of the material contacts the skin.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. The terms “including” and “such as” are not limiting and should be interpreted as “including, but not limited to,” and “such as, for example,” respectively. Moreover, any combination of the above-described embodiments in all possible variations thereof is encompassed by the disclosure unless otherwise indicated herein or otherwise clearly contradicted by context.

We claim:

1. An applicator for creating an electric field on the skin of a patient, the applicator comprising:
a substrate configured to receive a plurality of discrete reservoirs of a half cell metal in spaced and electrically isolated relation, the substrate defining insulating separations between neighboring reservoir locations (in a particular embodiment, by defining a plurality of ridges and troughs);
wherein neighboring reservoirs are electrically isolated from one another by an insulating separation such that when the applicator is applied to the patient the reservoirs form half-cells of a battery and the skin forms another half-cell of the battery that creates an electric field.

2. The applicator of claim 1 wherein the substrate defines a plurality of ridges and troughs and the reservoirs are deposited on the ridges with the troughs providing the insulating separation.

3. The applicator of claim 1, wherein the substrate is grown from a living organism.

4. The applicator of claim 1, wherein the substrate is collagen.

5. The applicator of claim 1, wherein the substrate is a textile.

6. The applicator of claim 1, wherein the reservoirs consist of zinc particles that are deposited onto the substrate.

7. A method of providing a half cell battery to a patient, the method comprising the acts of:
providing a substrate configured with insulating separations between reservoirs of a half-cell locations;
depositing reservoirs of a half-cell metal onto the substrate in a spaced arrangement such that neighboring reservoirs are electrically isolated from one another;
applying the substrate with the deposited zinc reservoirs to the patient such that the zinc reservoirs form half cells of a battery that creates an electric field.

8. The method of claim 7, further comprising forming a plurality of ridges and troughs in the substrate and depositing the reservoirs on the ridges with the troughs providing the insulating separation wherein the reservoirs are zinc particles.

9. The method of claim 8 further comprising solvent casting the reservoirs onto a plurality of the plurality of troughs.

10. The method of claim 7 wherein the substrate is collagen.

11. The method of claim 9 wherein the reservoirs are a zinc powder solvent casted onto the substrate by suspending the zinc powder in a liquid to create a slurry and dropping or spraying the slurry onto the substrate.

12. The method of claim 6 wherein the substrate releases oxygen over time.

13. The method of claim 13 wherein the substrate is a hydrogel, hydrocolloid, or starch.

14. The method of claim 8 further comprising suspending the zinc in a cellulose solution.

15. The method of claim 10 further comprising placing the substrate with the deposited zinc reservoirs on a location on the patient where collagen supplementation is desired.

16. A method of treating a condition, wherein the condition comprises at least one of purpura, joint pain, tennis elbow, convalescence aches, diabetic neuropathy, and arthritic conditions, the method comprising the acts of:
forming a plurality of fibers having a plurality of a half-cell metal embedded in the plurality of fibers;
forming a textile from the plurality of fibers
applying the textile to the skin of a patient in the vicinity of the condition to initiate galvanic reaction with the zinc reservoirs to create an electric field.

17. The method of claim 16 wherein the textile is at least one of a sleeve, an armband, a leg band, a sock, a shirt, shorts, or pants, any of which may be a compression garment or a non-compression garment.

18. The method of claim 16 further comprising forming a conductive network of carbon nanotubes in the fibers.

19. The method of claim 18, wherein the carbon nanotubes comprise between 0.1% and 10% by weight of the fibers.

20. The method of claim 18, wherein the fibers comprise one or more materials selected from the group consisting of polycellulose, carboxymethylcellulose, hydroxypropyl cellulose, alginate, chitosan, collagen, gelatin, hyaluronic acid, polyvinyl alcohol, polylactic acid, and combinations thereof.
